(19) United States
(12) Patent Application Publication
(10) Pub. No.: US 2014/0314825 A1
(43) Pub. Date: Oct. 23, 2014

(54) CHITOSAN DENTAL SURGICAL MEMBRANE AND METHOD OF MAKING

(71) Applicant: AGRATECH INTERNATIONAL, INC., Kinnelon, NJ (US)

(72) Inventors: Joseph F. Bristow, Lafayette, LA (US); Bruno R. Stockinger, Cliffside Park, NJ (US)

(21) Appl. No.: 14/359,254

(22) PCT Filed: Nov. 20, 2012
(86) PCT No.: PCT/US2012/066060

§ 371 Date: May 19, 2014

Related U.S. Application Data
(60) Provisional application No. 61/562,246, filed on Nov. 21, 2011.

(51) Int. Cl. A61L 31/14; A61L 31/04

(52) U.S. Cl. CPC A61L 31/146; A61L 31/042; A61L 2430/12
USPC 424/426; 427/2.1

(57) ABSTRACT

A porous, resorbable and flexible dental surgical membrane (16) is made from chitosan having a molecular weight of at least 400,000 daltons and has a thickness of from about 100 microns to about 0.5 mm. The membrane is easily insertable over a bone graft material site to confine the bone graft material (14) while allowing access to it of blood and oxygen. The high molecular weight of the chitosan may be chosen so that the membrane will not dissolve or resorb in a human mouth for a protracted period, e.g., from about 12 to about 16 weeks. The membrane is made by dissolving medical grade chitosan in aqueous acetic acid, dispersing fine silica particles into the solution to form a slurry, depositing a film of the slurry on a support surface, evaporating liquid from the slurry sufficiently to form a coherent chitosan membrane having silica particles dispersed therein, and then dissolving the silica particles with a sodium hydroxide solution followed by a water wash to form the porous chitosan membrane.

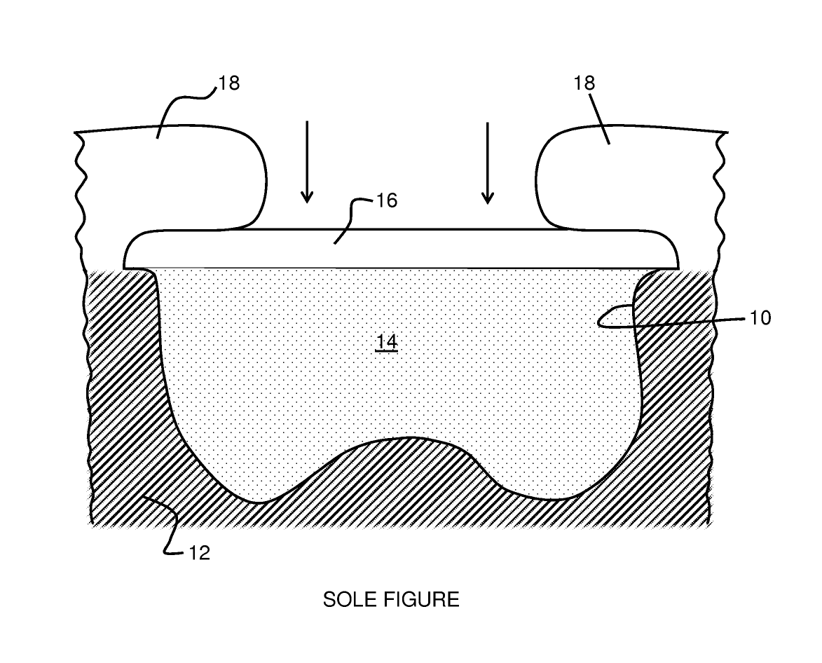

### CROSS-REFERENCE TO RELATED APPLICATION

This application claims the benefit of priority of provisional patent application Ser. No. 61/562,246, filed on Nov. 21, 2011, entitled “Resorbable Dental Surgical Membrane Made From Chitosan For Use In Oral Surgery”.

### BACKGROUND OF THE INVENTION

1. Field of the Invention

The present invention concerns a resorbable dental surgical membrane made from chitosan, which is well suited to enclose and protect granular bone graft material for a period of time sufficient to allow the bone graft material to develop into host bone in a patient.

2. Related Art

Bone graft material is granular in form and must be held in place in an opening, usually a surgically-constructed opening in a patient's bone structure until the granular bone graft material matures into monolithic host bone into which a structure such as a dental implant may be securely implanted. For example, in oral and implant surgery, after a tooth is extracted, bone graft material is inserted into the empty socket in the jawbone and eventually becomes a solid, monolithic bone suitable to secure a prosthetic tooth implanted therein. Bone graft material is also utilized during osseous alveolar and sinus augmentation procedures, which enhance bone structure. Known dental surgical materials used in oral and implant surgery are often stiff and inflexible, making them difficult to use, for example, difficult to insert under the gingiva around the bone graft material. Further, many of the known dental surgical materials are very expensive and often require an additional surgical procedure to remove the material once the bone graft has matured and before the prosthetic implant can be inserted into the matured bone graft.

U.S. Pat. No. 5,993,661, issued Nov. 30, 1999 to Eli Ruckenstein et al. for “Macroporous or Microporous Filtration Membrane, Method of Preparation and Use”, discloses microporous or macroporous affinity filtration membranes wherein the matrix is composed of chitosan or chitin and pores are made in the membrane by dissolution of a porogen during the preparation of the membrane. The patent also discloses a method of preparation of the membrane comprising preparing an acidic chitosan solution containing porogen, shaping the suspension into a membrane, and dissolving the porogen by immersing the membrane in an alkaline solution. The special feature of the membrane is that the pore size can be controlled by varying the size of the porogen. The membranes are said to be suitable for affinity purification of macromolecules.

Hemostatic bandages coated with chitosan are known and are available, for example, from HemCon Medical Technologies, Inc. of Portland, Oreg. 97223. That same company also supplies a family of dental dressings made of chitosan which is applied to bleeding oral surgical wounds to provide hemostasis by the attraction of red blood cells to the chitosan. As shown in a HemCon® dental dressing brochure disseminated by Zimmer Dental of Carlsbad, Calif., the HemCon® chitosan dental dressing is stated to be self-adhesive so that it does not require sutures and that it will usually dissolve in the mouth within 48 hours, thereby obviating the need for removal and leaving the site free of foreign material to allow natural wound healing.

### SUMMARY OF THE INVENTION

In accordance with the present invention, there is provided a flexible dental surgical membrane which comprises a porous chitosan membrane having a thickness of from about 100 microns to about 0.5 mm, and pores which render the membrane permeable to atmospheric oxygen and normal human red blood cells in the environment of a human mouth. The chitosan has a molecular weight of at least about 400,000 daltons, for example, from about 400,000 daltons to about 2,000,000 daltons, or from about 400,000 to about 800,000 daltons.

In one aspect of the present invention, the chitosan has a molecular weight high enough that the membrane emplaced in a human mouth will not be resorbed for a period of from about 12 to about 16 weeks.

Other aspects of the present invention include one or more of the following features, alone or in any suitable combination. The membrane may have a pore size distribution such that at least about ninety percent, preferably at least about 95 percent, of the pores are from about 10 to about 20 microns in diameter; and the chitosan comprises a medical grade chitosan.

A method aspect of the present invention comprises making a flexible dental surgical membrane and comprises the following steps. (a) Chitosan having a molecular weight of at least about 400,000 daltons is dissolved in an acidic aqueous solution, for example, an acetic acid solution. (b) Porogen particles are added to the acidic aqueous solution to form a slurry of solid porogen particles dispersed in a liquid phase comprising the acidic aqueous solution of the chitosan, at least ninety percent by weight, preferably at least 95 percent by weight, of the porogen particles having a diameter of from about 10 to about 20 microns. (c) The slurry is spread in a layer on a support surface to provide a slurry layer and the liquid phase is evaporated from the slurry layer sufficiently to leave behind a coherent chitosan membrane having the porogen particles distributed through the membrane. (d) The chitosan membrane is then contacted with a solvent to dissolve the porogen particles to leave pores in the spaces formerly occupied by the porogen particles. The pores are distributed through the chitosan membrane and have a pore size range substantially corresponding to that of the porogen particles, and the resulting chitosan membrane has a thickness of from about 100 microns to about 0.5 mm; and (e) rinsing the membrane to remove solvent residue from the membrane.

The present invention also provides one or more of the following aspects, alone or in any suitable combination. The porogen may comprise silica particles, the acidic aqueous solution may be a solution of acetic acid, the solvent may comprise an aqueous solution of sodium hydroxide; and the rinsing of step (e) may be carried out with water, preferably distilled water, as the rinsing agent; the solution of acetic acid may be a 1% (v/v) aqueous solution of acetic acid; in step (c), evaporating the liquid phase from the slurry layer may comprise allowing the slurry layer to be exposed to ambient atmosphere for a time sufficient to evaporate the liquid phase sufficiently to form the coherent chitosan membrane; during such evaporation the slurry layer may be heated in an ambient atmosphere at a temperature ranging from ambient temperature, e.g., about 20° C., up to not more than about 110° C., to evaporate the liquid phase.

As used herein and in the claims, “porogen” has its usual meaning of a particulate material which may be dispersed, usually into the liquid precursor of solid structures, and, after formation of the solid structure, may be dissolved or otherwise removed from the structure to leave pores in the spaces formerly occupied by the porogen.

### BRIEF DESCRIPTION OF THE DRAWINGS

The sole FIGURE of the drawings is a schematic cross-sectional view of a bone graft site in the jaw bone of a patient.

### DETAILED DESCRIPTION OF THE INVENTION AND SPECIFIC EMBODIMENTS THEREOF

Bone graft material is utilized in oral implant surgery and in osseous alveolar and sinus augmentation procedures and is available from numerous sources including, in the United States of America, Exactech Biologics of Painesville, Fla., Zimmer Dental of Carlsbad, Calif. (which supplies Geistlich Bio-Oss® bone graft material), Impladent Ltd. of Holliswood, N.Y. (which supplies Osteogen® bone graft material, and numerous other suppliers. The bone graft material is granular or powder-like, so that it may be packed into irregularly shaped sockets, cavities or other sites in bone formations. The granular bone graft material therefore must be held in place in a socket or other site in the patient's bone until it matures into a solid plug of host bone into which an implant such as a prosthetic tooth can be secured. To facilitate true bone formation, nutrients, oxygen and blood must be able to access the bone graft material, while preventing the soft tissue/gingival around the site from growing into the bone graft. The resorbable dental surgical chitosan membrane in accordance with the present invention prevents the bone graft material from migrating out of the bone graft site while allowing oxygen, blood and nutrients to access the bone graft material.

Blood, oxygen and nutrients should be able to access the bone graft material to facilitate true bone formation, but the surrounding soft tissue should be initially denied access to the bone graft to prevent ingrowth. Despite this, the surrounding soft tissue should eventually be allowed to grow over and cover the surgical site.

The chitosan membrane of the present invention differs significantly from known bone graft surgical membranes. The chitosan membrane of the present invention is flexible, making it easy to place over the bone graft material and tuck under the surrounding soft tissue, and is strong enough to contain the granular bone graft material. Further, the membrane of the present invention is porous so that it permits blood and oxygen as well as nutrients to access the bone graft material. In addition, while the chitosan-based membrane of the present invention resorbs in the mouth, it does so only after a period long enough to ensure that the bone graft material has matured into solid host bone. This characteristic reduces the amount of surgery required by eliminating the need for a second surgical procedure to remove the membrane, thereby reducing both the number of procedures to which a patient is subjected and the cost of oral surgery. Significantly, the chitosan membrane of the present invention, unlike the above-described HemCon® dental dressing, does not dissolve in the mouth within 48 hours, but rather will endure for weeks before it resorbs. The slow-resorbing characteristic is attained by using chitosan of at least 400,000 daltons molecular weight. The higher the molecular weight of the chitosan, the longer it will take for the membrane to dissolve or resorb. Chitosan is non-toxic and non-allergenic allowing for broad compatibility.

One embodiment of the membrane of the present invention is made from medical grade chitosan of molecular weight of at least 400,000 daltons, for example, from 400,000 daltons to 2,000,000 daltons, or from 400,000 daltons to 800,000 daltons, or any suitable molecular weight range lying within the range of 400,000 to 2,000,000 daltons. For example, a molecular weight range of the chitosan may be from about 450,000 or 500,000 daltons to about 800,000 or 1,000,000 daltons. The chitosan may be provided from any suitable source. For example, see U.S. Pat. No. 8,318,913, issued to Joseph Bristow, for “Chitosan Manufacturing Process”. Medical grade chitosan such as that obtainable by the process of the aforesaid U.S. Pat. No. 8,318,913, the disclosure of which is incorporated herein, is suitable for the uses of the present invention. Chitosan comprises deacetylated chitin; usually at least 70%, e.g., 70% to 95%, of the acetyl groups of chitin are removed and replaced, usually by amine groups, to provide chitosan.

### EXAMPLE

Medical grade chitosan of molecular weight of at least 400,000 daltons is dissolved in a sufficient amount of a 1% acetic acid aqueous solution to form a solution containing 1% by weight chitosan in grams per liter of solution (“w/v”). Silica of a particle size such that at least 90% by weight, preferably at least about 95% by weight, more preferably at least about 98% by weight, of the particles are between from about 10 microns to about 20 microns in diameter is used. A weight of the silica particles equal in weight to the chitosan is dispersed in the chitosan solution. The solution is spread on a rimmed glass plate such that a film of the slurry which will yield upon evaporation of the liquid from the slurry will leave behind a membrane of approximately 0.5 mm thickness. The membrane is allowed to dry and is then submersed in a 1 M sodium hydroxide solution at 75° C. for 2 hours to dissolve the silica, creating pores in the film corresponding to the spaces in the film previously occupied by the silica particles. The resulting porous membrane is washed with distilled water to remove the sodium hydroxide solution and allowed to dry. The silica particles are dispersed in the chitosan solution with stirring and a film of the resulting chitosan solution/silica particles slurry is deposited on a glass plate. The liquid component is evaporated from the slurry to leave behind a chitosan film. The resulting film is then submersed in a 1 M sodium hydroxide solution at 75° C. for 2 hours to dissolve the silica to form open-cell pores in the film.

The resulting pores in the chitosan film allow the migration of red blood cells and oxygen to the bone graft material, promoting better healing. Red blood cells typically have a diameter of from about 6 to about 8 microns. Chitosan is biodegradable and a polysaccharide so it will be slowly digested in the mouth during the healing process over a period of time commensurate with the molecular weight of the chitosan. For example, digestion or resorb time is preferably a period of weeks so that the membrane is resorbed by the time maturation of the bone graft is complete. It has been found that the higher the molecular weight, the longer it takes for the chitosan membrane to resorb, i.e., be digested or dissolved, in the mouth of a human being. The high molecular weight also provides a mechanically sturdier membrane.

The membrane may be cut to a desired size and shape and is then placed over powdered bone graft material disposed, for example, in a socket formed in the alveolar ridge of a patient's mouth. The edges of the membrane are tucked beneath the gingiva or soft tissue surrounding the site. The surrounding soft tissue is then sutured together over the membrane.

The porous chitosan membrane is easy to insert, confines the granular bone graft material, allows access of required substances, i.e., blood and atmospheric oxygen and any applied liquids such as bone nutrients, to the bone graft material, excludes unwanted substances from the bone graft material, allows soft tissue coverage of the site without in-growth of the soft tissue into the bone graft material, and resorbs over a period of time, e.g., 12 to 16 weeks, which is long enough to permit the bone graft material to mature into solid, i.e., monolithic as distinguished from granular or powder-like, host bone. Because the membrane eventually is resorbed, it reduces the amount of surgery required, and reduces the cost and morbidity of oral and implant surgery.

The sole FIGURE schematically shows a dental alveolus or tooth socket 10 in the jaw bone 12 of a patient. The tooth socket 10 is filled with an initially granular bone graft material 14 which is encased within tooth socket 10 by a chitosan membrane 16 which is overlapped on its opposite sides by the patient's gingiva 18. The porous chitosan membrane in accordance with the present invention is sufficiently porous to admit blood, atmospheric oxygen and any applied medicaments through the membrane 16 as indicated by the unmarked arrows in the FIGURE and into contact with bone graft material 14. The porous chitosan membrane 16 will eventually be resorbed but not until a period of from about 12 to 16 weeks has elapsed, during which time the initially granular bone graft material will mature into a solid host bone capable of receiving and securing a prosthetic tooth or the like mounted therein. Without wishing to be bound by any theory, it is believed that in the environment of a patient's mouth the chitosan membrane will eventually dissolve and so will effectively be resorbed to leave the mature bone graft material 14, now comprising a host bone, accessible for implantation of a prosthetic device therein.

Although the invention has been described with reference to human patients and certain characteristics of the surgical membrane refer to the environment of the human mouth, the membrane of the present invention also has utility in veterinary procedures carried out on a wide range of animals including dogs, cats, horses and zoo animals including elephants, camels, buffalo, etc.

1. A flexible dental surgical membrane comprises a chitosan membrane having a thickness of from about 100 microns to about 0.5 mm, and pores which render the membrane permeable to atmospheric oxygen and normal human red blood cells in the environment of a human mouth, the chitosan having a molecular weight of at least 400,000 daltons.

2. The surgical membrane of claim 1 wherein the chitosan has a molecular weight high enough that the membrane will not be resorbed for a period of from about 12 to about 16 weeks.

3. The surgical membrane of claim 1 wherein the chitosan has a molecular weight of from about 400,000 to about 2,000,000 daltons.

4. The surgical membrane of claim 1 wherein the chitosan has a molecular weight of from about 400,000 to about 800,000 daltons.

5. The surgical membrane of any one of claim 1, 2 or 3 wherein the membrane has a pore size distribution such that at least about ninety percent of the pores are from about 10 to about 20 microns in diameter.

6. The surgical membrane of claim 5 wherein at least about 95 percent by weight of the pores are from about 10 to about 20 microns in diameter.

7. The surgical membrane of any one of claim 1, 2 or 3 wherein the chitosan comprises a medical grade chitosan.

8. A method of making a flexible dental surgical membrane comprises the steps of:
(a) dissolving chitosan having a molecular weight of at least about 400,000 daltons in an acidic aqueous solution;
(b) adding porogen particles to the acidic aqueous solution to form a slurry of solid porogen particles dispersed in a liquid phase comprising the acidic aqueous solution of the chitosan, at least ninety percent by weight of the porogen particles having a diameter of from about 10 to about 20 microns;
(c) spreading the slurry in a layer on a support surface to provide a slurry layer and evaporating the liquid phase from the slurry layer sufficiently to leave behind a coherent chitosan membrane having the porogen particles distributed through the membrane;
(d) contacting the chitosan membrane with a solvent to dissolve the porogen particles to leave pores in the spaces formerly occupied by the porogen particles, the pores being distributed through the chitosan membrane and having a pore size range substantially corresponding to that of the porogen particles, the resulting chitosan membrane having a thickness of from about 100 microns to about 0.5 mm; and
(e) rinsing the membrane to remove solvent residue from the membrane.

9. The method of claim 8 wherein the porogen comprises silica particles, the acidic aqueous solution is a solution of acetic acid, the solvent comprises an aqueous solution of sodium hydroxide and the rinsing of step (e) is carried out with water as the rinsing agent.

10. The method of claim 8 or claim 9 wherein the solution of acetic acid is a 1% by volume (“v/v”) aqueous solution of acetic acid.

11. The method of claim 10 wherein the sodium hydroxide solution comprises a 1 M solution of sodium hydroxide.

12. The method of claim 8 wherein in step (c), evaporating the liquid phase from the slurry layer comprises allowing the slurry layer to be exposed to ambient atmosphere for a time sufficient to evaporate the liquid phase sufficiently to form the coherent chitosan membrane.

13. The method of claim 12 wherein the slurry layer is heated in an ambient atmosphere at a temperature of not more than about 110° C. to evaporate the liquid phase.
